(19) United States
(12) Patent Application Publication
(10) Pub. No.: US 2022/0015902 A1
(43) Pub. Date: Jan. 20, 2022

(54) Transcatheter Retrieval of Mitral or Tri-Cuspid Valves

(71) Applicant: Tendyne Holdings, Inc., St. Paul, MN (US)

(72) Inventor: Zachary J. Tegels, Minneapolis, MN (US)

(73) Assignee: Tendyne Holdings, Inc., St. Paul, MN (US)

(21) Appl. No.: 17/374,310

(22) Filed: Jul. 13, 2021

Related U.S. Application Data
(60) Provisional application No. 63/052,142, filed on Jul. 15, 2020.

(51) Int. Cl. A61F 2/24

(52) U.S. Cl. CPC A61F 2/2436; A61F 2002/9528

(57) ABSTRACT

An apparatus for collapsing a prosthetic heart valve and retrieving the prosthetic heart valve from a native heart valve annulus includes a delivery tube having a lumen therethrough and a retrieval device extendable from the lumen. The retrieval device includes a first shaft including a plurality of first arms selectively moveable between a collapsed condition and an expanded condition, and a second shaft slidable relative to the first shaft, the second shaft including a plurality of second arms selectively moveable between a collapsed condition and an expanded condition. The first and second arms in the expanded condition grasp portions of the prosthetic heart valve, and in the collapsed condition collapse the prosthetic heart valve for removal through the delivery tube.

### CROSS-REFERENCE TO RELATED APPLICATIONS

This application claims the benefit of the filing date of U.S. Provisional Patent Application No. 63/052,142 filed on Jul. 15, 2020 the disclosure of which is hereby incorporated herein by reference.

### BACKGROUND OF THE DISCLOSURE

The present disclosure relates to collapsible prosthetic heart valves, and more particularly, to apparatus and methods for retrieving improperly positioned or malfunctioning prosthetic heart valves after implantation.

Prosthetic heart valves that are collapsible to a relatively small circumferential size can be delivered into a patient less invasively than valves that are not collapsible. For example, a collapsible valve may be delivered into a patient via a tube-like delivery apparatus such as a catheter, a trocar, a laparoscopic instrument, or the like. This collapsibility can avoid the need for a more invasive procedure such as full open-chest, open-heart surgery.

Collapsible prosthetic heart valves typically take the form of a valve structure mounted on a stent. There are two types of stents on which the valve structures are ordinarily mounted: a self-expanding stent and a balloon-expandable stent. To place such valves into a delivery apparatus and ultimately into a patient, the valve must first be collapsed to reduce its circumferential size.

When a collapsed prosthetic valve has reached the desired implant site in the patient (e.g., at or near the native annulus of the patient's heart valve that is to be repaired by the prosthetic valve), the prosthetic valve can be deployed or released from the delivery apparatus and expanded to its full operating size. For balloon-expandable valves, this generally involves releasing the entire valve, assuring its proper location, and then expanding a balloon positioned within the valve stent. For self-expanding valves, on the other hand, the stent automatically expands as the stent is withdrawn from the delivery apparatus.

The clinical success of collapsible heart valves is dependent, in part, on the accurate positioning of the valve within the native valve annulus. Inaccurate placement and/or anchoring of the valve may result in the leakage of blood between the prosthetic heart valve and the native valve annulus. This phenomena is commonly referred to as paravalvular leakage. In mitral valves, paravalvular leakage enables blood to flow from the left ventricle back into the left atrium during systole, resulting in reduced cardiac efficiency and strain on the heart muscle.

Despite the various improvements that have been made to transcatheter mitral valve repair devices, various shortcomings remain. For example, due to the anatomy of the heart, it can be difficult to correctly align the prosthetic heart valve within the native mitral valve annulus of the patient during deployment of the prosthetic heart valve. Moreover, the prosthetic heart valve may “jump” or become repositioned during deployment as the stent of the prosthetic heart valve engages with tissue. In these instances, when the valve has been improperly deployed or has moved to an improper position during deployment, the prosthetic heart valve would need to be entirely removed from the patient. Removing a prosthetic heart valve after implantation typically requires surgery and greatly increases the risk of damaging the surrounding tissue of an already at risk patient.

Therefore, there is a need for a minimally invasive apparatus to safely and effectively remove an improperly positioned or malfunctioning prosthetic heart valve.

### BRIEF SUMMARY OF THE DISCLOSURE

In accordance with a first aspect of the present disclosure, a transcatheter retrieval system is provided for removing a malfunctioning or mispositioned collapsible prosthetic heart valve from a native valve annulus of a patient. Among other advantages, the retrieval system allows the prosthetic heart valve to be retrieved in a minimally invasive manner, avoiding the need for full open-chest, open-heart surgery.

One embodiment of the retrieval system includes a delivery tube having a lumen and a retrieval device extendable from the lumen for retrieving a prosthetic heart valve from a native valve annulus. The retrieval device includes a first shaft and a plurality of first arms hingedly mounted about the first shaft and selectively moveable between a collapsed condition and an expanded condition, and a second shaft, slidable relative to the first shaft, with a plurality of second arms hingedly mounted about the second shaft and selectively moveable between a collapsed condition and an expanded condition.

Another embodiment of the retrieval device includes a proximal shaft having a plurality of proximal arms hingedly mounted about the proximal shaft and selectively moveable between a collapsed condition and an expanded condition, and a distal shaft, slidable relative to the proximal shaft, with a plurality of distal arms hingedly mounted about the distal shaft and selectively moveable between a collapsed condition and an expanded condition. When the proximal arms and the distal arms are in the collapsed condition, the retrieval device may be extendable through the lumen of a delivery tube, and when the proximal arms and the distal arms are in the expanded condition, the proximal arms are configured to capture a first end of a prosthetic heart valve and the distal arms are configured to capture a second end of the prosthetic heart valve.

A method of retrieving a collapsible prosthetic heart valve from a native heart valves annulus is provided herein and initially includes extending a retrieval device out from a distal end of a lumen of a delivery tube toward the prosthetic heart valve to be retrieved. The retrieval device may include a first shaft, and a plurality of first arms hingedly mounted about the first shaft and selectively moveable between a collapsed condition and an expanded condition, and a second shaft, and a plurality of second arms hingedly mounted about the second shaft and selectively moveable between a collapsed condition and an expanded condition. The method further includes, sliding the second shaft relative to the first shaft and through a prosthetic heart valve implanted within a native annulus of a patient, capturing a first end of the prosthetic heart valve using the plurality of first arms and capturing a second end of the prosthetic heart valve using the plurality of second arms, transitioning the plurality of first arms and the plurality of second arms from the expanded condition to the collapsed condition, retracting the retrieval device and the prosthetic heart valve into the lumen of the delivery tube and withdrawing the delivery tube from the patient.

### BRIEF DESCRIPTION OF THE DRAWINGS

Various embodiments of the present disclosure are described herein with reference to the drawings, wherein:

FIG. 1 is a highly schematic cutaway view of the human heart, showing two approaches for delivering a prosthetic mitral valve to an implantation site;

FIG. 2 is a highly schematic representation of a native mitral valve and associated cardiac structures;

FIG. 3A is a side view of a prosthetic mitral valve according to the prior art;

FIG. 3B is a longitudinal cross-sectional view of the prosthetic mitral valve of FIG. 3A;

FIG. 4 is a highly schematic, partial side elevational view of a transcatheter prosthetic heart valve retrieval system in accordance with an embodiment of the present disclosure;

FIGS. 5-7 are highly schematic longitudinal elevational views showing the removal of a mispositioned prosthetic mitral valve from the native mitral valve annulus of a patient using the transcatheter retrieval system of FIG. 4 and a transseptal approach.

### DETAILED DESCRIPTION

Blood flows through the mitral valve from the left atrium to the left ventricle. As used herein in connection with a prosthetic heart valve, the term “inflow end” refers to the end of the heart valve through which blood enters when the valve is functioning as intended, and the term “outflow end” refers to the end of the heart valve through which blood exits when the valve is functioning as intended. Further, when used herein in connection with a device for retrieving an implanted prosthetic valve, the terms “proximal” and “distal” are to be taken as relative to a user using the device in the intended manner. “Proximal” is to be understood as relatively close to the user and “distal” is to be understood as relatively farther away from the user. Also as used herein, the terms “substantially,” “generally,” and “about” are intended to mean that slight deviations from absolute are included within the scope of the term so modified.

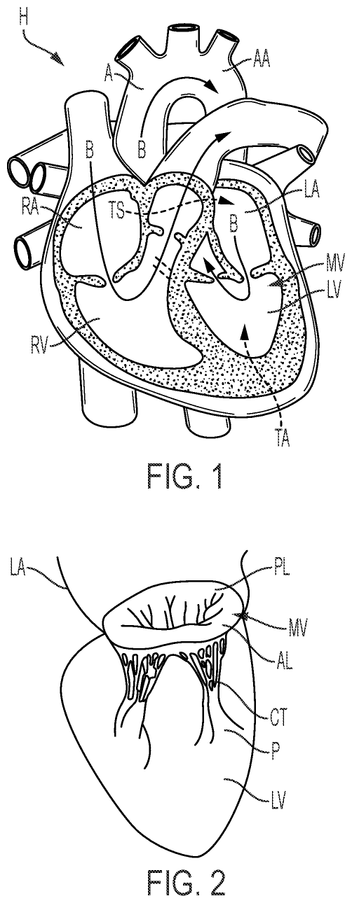

FIG. 1 is a schematic cutaway representation of a human heart H. The human heart includes two atria and two ventricles: right atrium RA and left atrium LA, and right ventricle RV and left ventricle LV. Heart H further includes aorta A and aortic arch AA. Disposed between the left atrium and the left ventricle is mitral valve MV. The mitral valve, also known as the bicuspid valve or left atrioventricular valve, is a dual-flap that opens as a result of increased pressure in left atrium LA as it fills with blood. As atrial pressure increases above that in left ventricle LV, mitral valve MV opens and blood flows into the left ventricle. Blood flows through heart H in the direction shown by arrows “B”.

A dashed arrow, labeled “TA”, indicates a transapical approach of implanting a prosthetic heart valve, in this case to replace the mitral valve. In the transapical approach, a small incision is made between the ribs of the patient and into the apex of left ventricle LV to deliver the prosthetic heart valve to the target site. A second dashed arrow, labeled “TS”, indicates a transseptal approach of implanting a prosthetic heart valve in which the valve is passed through the septum between right atrium RA and left atrium LA. Other approaches for implanting a prosthetic heart valve are also possible and may be used in accordance with the present disclosure.

FIG. 2 is a more detailed schematic representation of native mitral valve MV and its associated structures. As previously noted, mitral valve MV includes two flaps or leaflets, posterior leaflet PL and anterior leaflet AL, disposed between left atrium LA and left ventricle LV. Cord-like tendons, known as chordae-tendineae CT, connect the two leaflets to the medial and lateral papillary muscles P. During atrial systole, blood flows from higher pressure in left atrium LA to lower pressure in left ventricle LV. When left ventricle LV contracts during ventricular systole, the increased blood pressure in the chamber pushes the posterior and anterior leaflets to close, preventing the backflow of blood into left atrium LA. Since the blood pressure in left atrium LA is much lower than that in left ventricle LV, the leaflets attempt to evert to low pressure regions. Chordae tendineae CT prevent the eversion by becoming tense, thus pulling on the leaflets and holding them in the closed position.

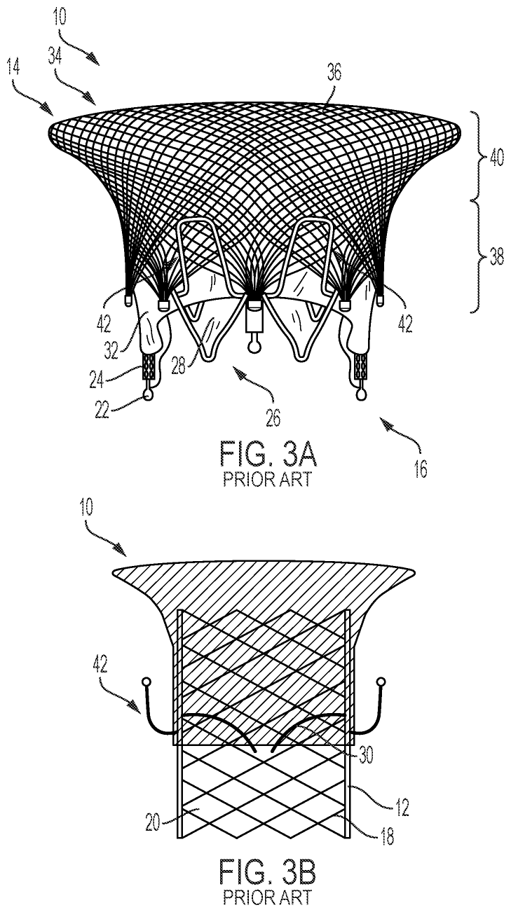

FIGS. 3A and 3B are a side view and a longitudinal cross-sectional view of prosthetic heart valve 10 according to the prior art. Prosthetic heart valve 10 is a collapsible and expandable prosthetic heart valve designed to replace the function of the native mitral valve MV (shown in FIGS. 1-2) of a patient. When used to replace native mitral valve MV, prosthetic valve 10 may have a low profile so as not to interfere with the heart's electrical conduction system pathways as well as atrial function.

Prosthetic heart valve 10 may include a stent 12, which may be formed from biocompatible materials that are capable of self-expansion, for example, shape-memory alloys such as nitinol. Alternatively, stent 12 may be balloon expandable or expandable by another force exerted radially outward on the stent. Stent 12 has a substantially cylindrical shape with an inflow end 14 and an outflow end 16. Stent 12 may include a plurality of struts 18 that form cells 20 connected to one another in one or more annular rows circumferentially extending about the stent. In one example, stent 12 is formed by laser cutting a predetermined pattern into a metallic tube. Cells 20 may be substantially the same size around the perimeter of stent 12 and along the length of the stent. Alternatively, cells 20 near inflow end 14 may be larger than the cells near outflow end 16. When deployed at a target site (e.g., within a native mitral valve annulus), stent 12 may expand radially outwardly and provide a force against the native valve annulus to assist in stabilizing prosthetic heart valve 10 within the native mitral valve annulus.

A plurality of commis sure attachment features 24 may be provided on stent 12 for attaching the commissure between two adjacent leaflets to the stent. As shown in FIG. 3A, commissure attachment features 24 may lie between two adjacent cells positioned in the same annular row adjacent the outflow end 16 of stent 12. Commissure attachment features 24 may include one or more eyelets that facilitate the suturing of the leaflet commissure to stent 12.

One or more retaining elements 22 may be provided at the outflow end 16 of stent 12. As shown in FIG. 3A, retaining elements 22 may extend from commissure attachment features 22. Retaining elements 22 are sized to cooperate with a corresponding retaining structure on a delivery device (not shown) for delivering prosthetic heart valve 10 into the patient. This cooperation minimizes the axial movement of prosthetic heart valve 10 relative to the delivery device during unsheathing or resheathing procedures, and minimizes rotation of the prosthetic heart valve relative to the delivery device as the delivery device is advanced to the target location and during deployment.

Prosthetic heart valve 10 also includes a valve assembly 26, which may be secured to stent 12 by suturing the valve assembly to struts 18 and/or to commissure attachment features 24. Valve assembly 26 includes a cuff 28 and a plurality of leaflets 30 that open and close collectively to function as a one-way valve. Both cuff 28 and leaflets 30 may be wholly or partly formed of any suitable biological material, such as bovine or porcine pericardium, or biocompatible polymer, such as polytetrafluorethylene (PTFE), urethanes and the like. A sealing skirt 32 may be disposed about an abluminal surface of stent 12. When prosthetic heart valve 10 is implanted within the native mitral valve annulus of a patient, sealing skirt 32 may seal any space between the prosthetic heart valve and the native mitral valve annulus to help prevent the backflow of blood into left atrium LA. Sealing skirt 32 may also be formed of any suitable biological material, such as bovine or porcine pericardium, or biocompatible polymer, such as PTFE, urethanes or similar materials.

Prosthetic heart valve 10 may also include a frame 34 positioned around stent 12 and valve assembly 26. Frame 34 may include a plurality of wires 36 or braided strands. Frame 34 may be formed from biocompatible materials that are capable of self-expansion, for example, shape-memory alloys, or from a balloon expandable or other mechanically expandable material, and may include a substantially cylindrical portion 38 disposed generally around the midsection of stent 12 and a flared portion 40 disposed adjacent the inflow end 14 of the stent. The flared portion 40 of frame 34 projects radially outward from stent 12 and is designed to at least partially project into the left atrium of the heart to anchor the stent within the native mitral valve annulus of the patient. The flared portion 40 of frame 34 may also help to fill voids between sealing skirt 32 and the native mitral valve annulus when prosthetic heart valve 10 is implanted in the native mitral valve annulus. In this manner, irregularities in the native mitral valve annulus may be filled by frame 34, thereby helping to prevent paravalvular leakage.

As shown in FIG. 3A, prosthetic heart valve 10 may also include one or more engagement arms 42 circumferentially mounted around stent 12 to engage tissue and stabilize the prosthetic heart valve within the native mitral valve annulus. Engagement arms 42 may be pivotally mounted to stent 12 and may be transitionable from a collapsed condition in which the engagement arms lie flush against collapsed stent 12 during delivery of prosthetic heart valve 10 into the patient, to an expanded condition in which the engagement arms extend radially outward from the stent after the prosthetic heart valve has been deployed from the delivery device.

Prosthetic heart valve 10 may be delivered to the desired site (e.g., at or near the native mitral valve annulus) by a delivery device (not shown) using a transapical, transseptal or another approach. Once prosthetic heart valve 10 has been properly positioned inside the native mitral valve annulus of the patient, it works as a one-way valve, allowing blood to flow into left ventricle LV, and preventing blood from returning to left atrium LA. If, however, prosthetic heart valve 10 is not accurately positioned within the native valve annulus, paravalvular leakage may occur between the prosthetic heart valve and the native mitral valve annulus despite the presence of sealing skirt 32 and the flared portion 40 of frame 34. Misplacement may occur as a result of the delivery device being misaligned with respect to the native mitral valve annulus when prosthetic valve 10 is deployed, or as a result of the prosthetic heart valve being repositioned as the valve contacts tissue. Because paravalvular leakage can reduce cardiac efficiency and strain the heart muscle, it is often necessary to remove an improperly positioned prosthetic heart valve from the native valve annulus of the patient after implantation.

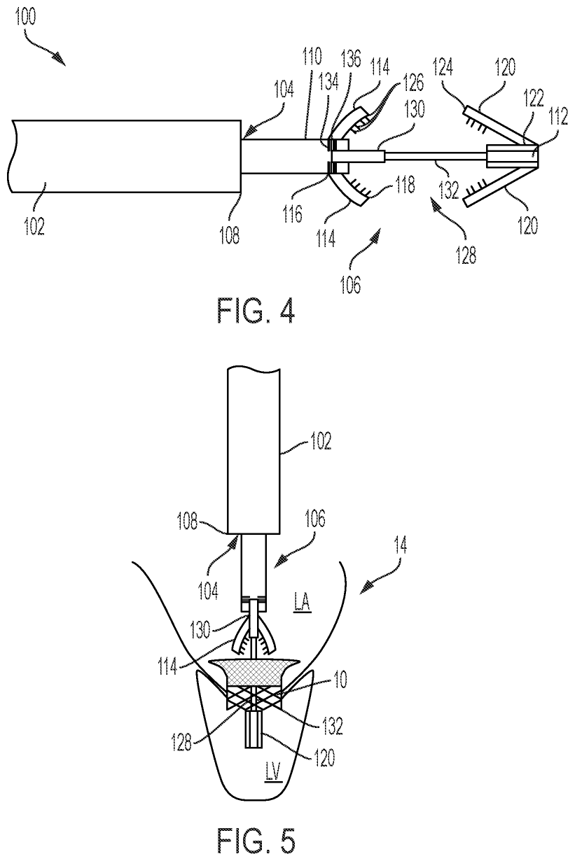

FIG. 4 is a partial, schematic illustration of a transcatheter system 100 for collapsing and removing a prosthetic heart valve from a native annulus of a patient according to one embodiment of the present disclosure. Transcatheter system 100 is designed to collapse and remove an implanted prosthetic heart valve, such as prosthetic mitral valve 10, from the native heart valve annulus of the patient in a minimally invasive manner. While transcatheter system 100 is primarily described herein as collapsing and removing prosthetic mitral valve 10, it will be appreciated that the transcatheter system may also be used to collapse and remove any prosthetic heart valve, including other bicuspid valves, or tricuspid valves, such as prosthetic aortic valves.

Transcatheter system 100 includes a handle (not shown) connected to a delivery tube 102 having a lumen 104 therethrough and a retrieval device 106 slidably disposed within the lumen of the delivery tube. The handle may include one or more actuators, such as rotatable knobs, linear slides, pull handles, levers, or buttons, for controlling the operation of retrieval device 106. Delivery tube 102 extends from a distal or leading end 108 to a proximal or trailing end (not shown) at which the delivery tube is connected to the handle. Delivery tube 102 may be any tube-like delivery device, such as a catheter, a trocar, a laparoscopic instrument, or the like, configured to house retrieval device 106 as the retrieval device is advanced toward the target site (e.g., the prosthetic heart valve to be removed).

Retrieval device 106 may be mounted on a carriage (not shown) that is housed within delivery tube 102 and operably coupled to the handle. The carriage is thus configured to control movement of the retrieval device into and out from the distal end 108 of delivery tube 102. By way of example, the carriage may be coupled to a first actuator, which may be a linearly translatable actuator such as a slider, for quickly translating the carriage and, in turn, retrieval device 106 into and out from the distal end 108 of delivery tube 102. The first actuator, however, may otherwise operate in any manner that allows retrieval device 106 to be moved relative to delivery tube 102, including proximal retraction of the delivery tube toward the handle to expose the retrieval device.

Retrieval device 106 may include a flexible first or proximal shaft 110 extending in a longitudinal direction and a flexible second or distal shaft 112 extending in the longitudinal direction. In one preferred embodiment, distal shaft 112 is slidable relative to proximal shaft 110.

A plurality of proximal arms 114 may be circumferentially mounted about proximal shaft 110. As shown in FIG. 4, retrieval device 106 may include two proximal arms 114 diametrically opposed to one another about proximal shaft 110. It will be appreciated, however, that retrieval device 106 may include any number of proximal arms 114 other than two, including three or more proximal arms. Each proximal arm 114 has an attached end 116 hingedly connected to proximal shaft 110 and extends distally toward free end 118. Proximal arms 114 may be selectively actuatable between a collapsed condition in which the free ends 118 of the arms lie close to or against proximal shaft 110, and an expanded condition in which the arms extend radially outward relative to the proximal shaft to surround and capture a prosthetic heart valve, such as prosthetic mitral valve 10. In the expanded condition, proximal arms 114 form a capture space that faces toward distal shaft 112. Proximal arms 114 may be transitioned between the collapsed condition and the expanded condition by manipulation of a second actuator (not shown) provided on the handle. In one preferred embodiment, the second actuator may be a knob coupled to a nut/lead-screw device with the lead-screw having a rigid section disposed within the handle and attached to a relatively flexible, yet torqueable shaft extending from the handle toward proximal arms 114. In this manner, the second actuator can precisely control movement of proximal arms 114 between the collapsed condition and the expanded condition. In a preferred embodiment, proximal arms 114 may be slightly curved (e.g., concave toward the longitudinal axis of retrieval device 106) to capture the flared inflow end 14 of prosthetic mitral valve 10 when the proximal arms are in the expanded condition.

A plurality of distal arms 120 may be circumferentially mounted about the distal shaft 112 of retrieval device 106. For example, retrieval device 106 may include two distal arms 120 diametrically opposed to one another about distal shaft 112, as shown in FIG. 4, or any number of distal arms other than two, including three or more distal arms. In some embodiments, the number of distal arms 120 is equal to the number of proximal arms 114. In other embodiments, the number of distal 120 arms may be less than or greater than the number of proximal arms 114. Each distal arm 120 has an attached end 122 hingedly connected to distal shaft 112 and extends proximally toward a free end 124. Distal arms 120 may be selectively actuatable between a collapsed condition in which the free ends 124 of the arms lie close to or against distal shaft 112, and an expanded condition in which the arms extend radially outward relative to the distal shaft to capture a prosthetic heart valve. In the expanded condition, distal arms 120 form a capture space that faces toward proximal shaft 110. Like proximal arms 114, distal arms 120 may be transitioned between the collapsed condition and the expanded condition using the second actuator, or a similarly constructed actuator, to precisely control movement of the distal arms. When proximal arms 114 and distal arms 120 are in the collapsed condition, the free ends 118 of the proximal arms extend substantially in a distal direction, and the free ends 124 of the distal arms extend substantially in a proximal direction such that retrieval device 106 has a diameter that allows the retrieval device to slide within the lumen 104 of delivery tube 102.

One or more engagement features 126 may be positioned on a surface of the proximal arms 114 and the distal arms 120 that faces the longitudinal axis of retrieval device 106. Engagement features 126 may be formed as a barb or other protrusion-like feature and may include a sharp tip to pierce the sealing skirt 32 of prosthetic mitral valve 10. Engagement features 126 may additionally be sized to pass through the cells 20 of the prosthetic mitral valve to firmly grasp the prosthetic mitral valve when proximal arms 114 and distal arms 120 are actuated from the expanded condition to the collapsed condition.

Retrieval device 106 may further include a telescoping extension 128 coupling proximal shaft 110 and distal shaft 112. Extension 128 may have a first or proximal portion 130 disposed within and connected to proximal shaft 110, and a second or distal portion 132 fixedly attached to distal shaft 112. The distal portion 132 of extension 128 may telescope into the proximal portion 130 of the extension to adjust the relative distance between proximal shaft 110 and distal shaft 112, and in turn, the relative distance between the proximal arms 114 and the distal arms 120 of retrieval device 106. For example, extension 128 may telescope from a retracted condition in which distal shaft 112 abuts or is otherwise proximate proximal shaft 110, to an extended condition in which the distal shaft is spaced a greater distance apart from the proximal shaft. Movement of distal shaft 112 relative to proximal shaft 110 may be controlled by a third actuator, for example, a pull handle that linearly translates the distal shaft toward and away from the proximal shaft relatively quickly. The pull handle may optionally also include a rotatable a knob for “fine tune” adjustments to precisely control the relative positioning of proximal shaft 110 and distal shaft 112.

In a preferred embodiment, the proximal portion 130 of extension 128 may include a protrusion 134 that is disposed within a groove 136 defined in an interior surface of proximal shaft 110 to rotatably couple extension 128, and with it, distal shaft 112, to the proximal shaft, while preventing translation of the proximal portion 130 relative to the proximal shaft. It will be appreciated, however, that any other coupling mechanism known in the art may be employed to rotatably couple distal shaft 112 to proximal shaft 110, and thus allow the distal arms 120 and the proximal arms 114 of retrieval device 106 to be independently positioned relative to prosthetic mitral valve 10. Additionally, distal portion 132 and proximal portion 130 may include corresponding flats (not shown) to telescopingly couple the distal portion within the proximal portion, while preventing unwanted rotational movement of distal shaft 112 relative to proximal shaft 110. In one example, the third actuator (e.g., the pull handle), may have a fixed range of rotation, for example, between about 15 degrees and about 30 degrees, to rotate extension 128 relative to proximal shaft 110 and modify the rotational orientation of distal arms 120 relative to proximal arms 114.

The use of transcatheter system 100 to remove an implanted, malfunctioning or mispositioned prosthetic heart valve from a native valve annulus of a patient will now be described with reference to FIGS. 5-7. A physician may initially manipulate the knob of the second actuator and transition the proximal arms 114 and the distal arms 120 of retrieval device 106 to the collapsed condition to load the retrieval device into the lumen 104 of delivery tube 102 such that the proximal arms and distal arms are entirely covered by the delivery tube. Transcatheter system 100 may then be percutaneously inserted into the patient and advanced toward the prosthetic heart valve to be removed. In removing prosthetic mitral valve 10 from the native mitral valve annulus using a transseptal approach, for example, the leading end 108 of delivery tube 102 may be guided into the left atrium LA of heart H.

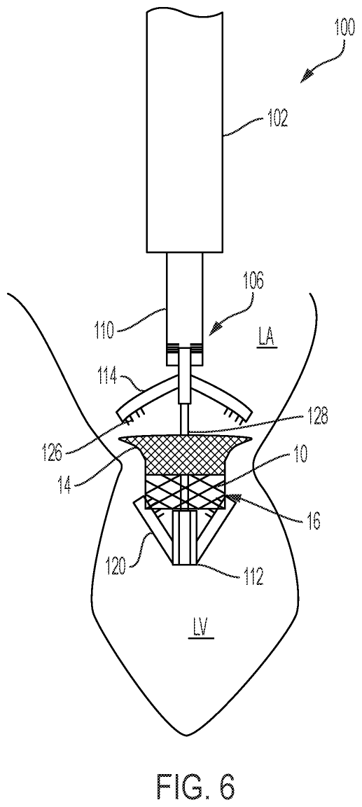

After the leading end 108 of delivery tube 102 has been positioned near prosthetic mitral valve 10, the user may slide the first actuator to extend retrieval device 106 from the lumen 104 of delivery tube 102. Referring to FIG. 5, with distal arms 120 in the collapsed condition, the user may manipulate the third actuator, for example, by pushing the pull handle in a distal direction, to slide distal shaft 112, and in turn, the distal arms 120 of retrieval device 106, away from proximal shaft 110, through prosthetic mitral valve 10 and into the left ventricle LV of heart H. The proximal arms 114 and the distal arms 120 of retrieval device 106 may then be transitioned, sequentially or simultaneously, from the collapsed condition to the expanded condition by manipulating the second actuator so as to create capture spaces that are sized to receive, respectively, the inflow end 14 of prosthetic mitral valve 10 and the outflow end 16 of the prosthetic mitral valve. Extension 128 may then be retracted slightly, via the fine tuning knob actuator on the pull handle, moving the distal shaft 112 closer to the proximal shaft 110 so that the inflow end 14 and outflow end 16 of prosthetic mitral valve 10 are positioned within the respective capture spaces, as shown in FIG. 6.

The curved shape of proximal arms 114 may aid in capturing the flared portion 40 of the frame 34 of prosthetic mitral valve 10. If the user cannot easily position the proximal arms 114 and/or the distal arms 120 of retrieval device 106 around the inflow end 14 and/or the outflow end 16 of prosthetic mitral valve 10 due to the misalignment of the prosthetic mitral valve relative to the native mitral valve annulus, the physician may optionally rotate the pull handle to rotate extension 128, and in turn distal arms 120, relative to proximal shaft 110, independently of proximal arms 114 such that the retrieval device is aligned in an optimal grasping position relative to the inflow and outflow ends of the prosthetic mitral valve.

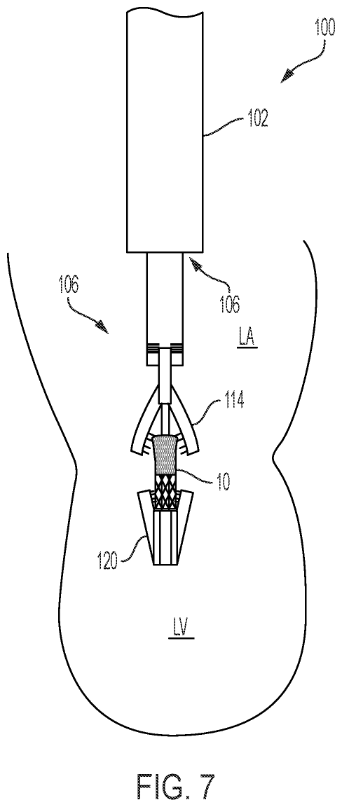

Referring to FIG. 7, the physician may then transition the proximal arms 114 of retrieval device 106 to the collapsed condition and, simultaneously or independently, may transition the distal arms 120 of the retrieval device to the collapsed condition, by manipulating the second actuator, causing the engagement features 126 of the proximal arms and the engagement features of the distal arms to pierce the sealing skirt 32 and pass through the cells 20 of prosthetic mitral valve 10. Further collapsing of the proximal arms 114 and the distal arms 120 of retrieval device 106 will cause prosthetic mitral valve 10 to collapse along its longitudinal axis. Retrieval device 106, along with prosthetic mitral valve 10, may then be retracted proximally, toward the user, and into the lumen 104 of delivery tube 102 by pulling the slide of the first actuator proximally, before the transcatheter system 100 is removed from the patient.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for collapsing a prosthetic heart valve, comprising:
a delivery tube having a lumen; and
a retrieval device extendable from the lumen, the retrieval device comprising:
a first shaft, and a plurality of first arms hingedly mounted about the first shaft and selectively moveable between a collapsed condition and an expanded condition; and
a second shaft slidable relative to the first shaft, and a plurality of second arms hingedly mounted about the second shaft and selectively moveable between a collapsed condition and an expanded condition.

2. The system of claim 1, wherein each of the first arms has an end attached to the first shaft and a free end, and when the first arms are in the collapsed condition, each of the ends of the first arms extends substantially in a distal direction from the attached end to the free end, and when the first arms are in the expanded condition, each of the free ends of the first arms extends radially away from the first shaft.

3. The system of claim 2, wherein each of the second arms has an end attached to the second shaft and a free end, and when the second arms are in the collapsed condition each of the free ends of the second arms extends substantially in a proximal direction from the attached end of the second arm to the free end of the second arm, and when the second arms are in the expanded condition, each of the free ends of the second arms extends radially away from the second shaft.

4. The system of claim 1, further comprising an extension having a first portion disposed within and connected to the first shaft, and a second portion attached to the second shaft, the extension being extendable from a first condition in which the second shaft is proximate the first shaft, and a second condition in which the second shaft is spaced apart from the first shaft.

5. The system of claim 4, wherein the first portion of the extension further includes a protrusion disposed within a groove defined in the first shaft such that the second shaft is rotatable relative to the first shaft.

6. The system of claim 1, wherein the plurality of first arms is circumferentially disposed about the first shaft and the plurality of second arms is circumferentially disposed about the second shaft.

7. The system of claim 1, wherein the plurality of first arms includes two first arms diametrically opposed to one another about the first shaft, and the plurality of second arms includes two second arms diametrically opposed to one another about the second shaft.

8. The system of claim 1, wherein each one of the plurality of first arms is curved concavely relative to a longitudinal axis of the retrieval device.

9. The system of claim 1, wherein at least one of the plurality of first arms and at least one of the plurality of second arms includes an engagement feature.

10. The system of claim 9, wherein the engagement feature comprises a protrusion.

11. A retrieval device for collapsing a prosthetic heart valve, comprising:
a proximal shaft, and a plurality of proximal arms hingedly mounted about the proximal shaft and selectively moveable between a collapsed condition and an expanded condition; and
a distal shaft slidable relative to the proximal shaft, and a plurality of distal arms hingedly mounted about the distal shaft and selectively moveable between a collapsed condition and an expanded condition,
wherein when the proximal arms and the distal arms are in the collapsed condition, the retrieval device is moveable through the lumen of a delivery tube, and when the proximal arms and the distal arms are in the expanded condition, the proximal arms are configured to capture a first end of a prosthetic heart valve and the distal arms are configured to capture a second end of the prosthetic heart valve.

12. The device of claim 11, further comprising an extension having a first portion disposed within and connected to the proximal shaft, and a second portion attached to the distal shaft, the extension being extendable from a first condition in which the distal shaft is spaced a first distance apart from the proximal shaft, and a second condition in which the distal shaft is spaced a second distance apart from the proximal shaft, the second distance being greater than the first distance.

13. The device of claim 11, wherein the plurality of proximal arms includes two proximal arms diametrically opposed to one another about the proximal shaft, and the plurality of distal arms includes two distal arms diametrically opposed to one another about the distal shaft.

14. The device of claim 11, wherein each of the proximal arms is curved to capture a flared end of a prosthetic heart valve.

15. The device of claim 11, wherein the distal shaft is rotatable relative to the proximal shaft.

16. A method of collapsing and removing a collapsible prosthetic heart valve from a native valve annulus of a patient, the method comprising:
inserting a leading end of a delivery tube into the patient proximate the prosthetic heart valve;
extending a retrieval device through a lumen of the delivery tube, the retrieval device comprising a first shaft and a second shaft, a plurality of first arms hingedly mounted about the first shaft and selectively moveable between a collapsed condition and an expanded condition, and a plurality of second arms hingedly mounted about the second shaft and selectively moveable between a collapsed condition and an expanded condition;
sliding the second shaft away from the first shaft and through a prosthetic heart valve implanted within a native annulus of a patient;
capturing a first end of the prosthetic heart valve using the plurality of first arms and capturing a second end of the prosthetic heart valve using the plurality of second arms;
transitioning the plurality of first arms from the expanded condition to the collapsed condition and transitioning the plurality of second arms from the expanded condition to the collapsed condition to transition the prosthetic heart valve from an expanded condition to a collapsed condition;
retracting the retrieval device and the prosthetic heart valve into the lumen of the delivery tube; and
withdrawing the delivery tube from the patient.

17. The method of claim 16, wherein the capturing step comprises:
engaging the first engagement feature with a first portion of the prosthetic heart valve and engaging the second engagement feature with a second portion of the prosthetic heart valve.

18. The method claim 17, wherein the engaging steps comprises piercing a sealing skirt of the prosthetic heart valve with the first and second engagement features.

19. The method of claim 17, wherein the first portion of the prosthetic heart valve is an inflow end of the prosthetic heart valve and the second portion of the prosthetic heart valve is an outflow end of the prosthetic heart valve.

20. The method of claim 16, further comprising:
rotating the second shaft relative to the first shaft to position the second arms relative to the prosthetic heart valve.
